(19) United States
(12) Patent Application Publication
(10) Pub. No.: US 2020/0093864 A1
(43) Pub. Date: Mar. 26, 2020

(54) NOVEL THERAPEUTIC STEM CELL COMPOSITIONS WITH AN ACTIVE OXIDATIVE PHOSPHORYLATION SITE AND THEIR PREPARATION

(71) Applicant: Kilmer S. McCully, Winchester, MA (US)

(72) Inventor: Kilmer S. McCully, Winchester, MA (US)

(21) Appl. No.: 16/138,898

(22) Filed: Sep. 21, 2018

(51) Int. Cl. A61K 35/28; A61K 31/7084

(52) U.S. Cl. CPC A61K 35/28; A61K 31/7084

(57) ABSTRACT

Novel modified stem cells activated as a carrier for thioretinaco and thioretinamide in the form of mitochondrial thioretinaco ozonide oxygen nicotinamide adenine dinucleotide phosphate are provided for the prevention and treatment of cancer, arteriosclerosis, dementia, autoimmune and other degenerative conditions involving increased levels of homocysteine. Methods for the production of the novel modified stem cells and their use are also provided.

### BACKGROUND OF THE INVENTION

### 1. Field of the Invention

This invention relates to an activated stem cell carrier for utilization of mitochondrial thioretinaco ozonide oxygen nicotinamide adenine dinucleotide phosphate (TR₂CoO₃O₂NAD⁺H₂PO₄⁻) in prevention and treatment of cancer, arteriosclerosis, osteoporosis, dementia, autoimmune disease, and diseases associated with higher than normal levels of homocysteine. Stem cells contain mitochondrial TR₂CoO₃O₂NAD⁺H₂PO₄⁻, the active site complex responsible for formation of adenosine triphosphate (ATP) from NAD⁺ and H₂PO₄⁻ by the process of oxidative phosphorylation. Exposure of stem cells to the active site complex within cell culture medium increases the concentration of the active site complex within mitochondria of stem cells, enhancing the ability of the activated stem cells to replace the active site complex which is depleted from mitochondria during aging, carcinogenesis, atherogenesis, and the pathophysiological processes involved in disease, aging and abnormal homocysteine metabolism.

### 2. Description of the Prior Art

Abnormal homocysteine metabolism was first implicated in the etiology of degenerative diseases by observation of accelerated arteriosclerosis in children with two different inherited enzymatic disorders resulting from deficiency of cystathionine synthase and methionine synthase, as reported by McCully K S in American Journal of Pathology 1969; 56:111-128. Accelerated arteriosclerosis was subsequently demonstrated in a child with deficiency of methylene tetrahydrofolate reductase, a third enzymatic disorder of homocysteine metabolism, as reported by Kanwar Y S et al in Pediatric Research 1976; 10:598-609. In all three of these enzymatic disorders, elevation of blood levels of homocysteine is implicated in the pathogenesis of arteriosclerosis by a direct effect of homocysteine on the metabolic activity of arterial cells and tissues.

Abnormal homocysteine thiolactone metabolism was demonstrated in cultures of cells from malignant tissues, as reported by McCully K S in Cancer Research 1976; 36:3198-3202. The results of this study show that cultured malignant cells contain a metabolic blockade of the oxidation of the sulfur atom of homocysteine thiolactone to sulfate, leading to accumulation of homocysteine thiolactone within malignant cells. Homocysteine thiolactone reacts with the free amino groups of macromolecules, forming peptide bonds that cause homocysteinylation of the amino groups of proteins, nucleic acids, and glycosaminoglycans. This metabolic blockade within malignant cells is ascribed to deficiency of a derivative of homocysteine thiolactone that normally occurs within non-malignant cells.

The formation of homocysteine thiolactone from methionine in malignant cells is catalyzed by methionyl t-RNA synthase by an error editing reaction, as reported by Jakubowski H et al in FEBS Letters 1993; 317:237-240. Abnormal homocysteine thiolactone metabolism in malignant cells is hypothesized to result from a deficiency of or a failure to synthesize an N-substituted derivative of homocysteine thiolactone, as discussed by McCully K S in Cancer Research 197636:2198-3202. According to this hypothesis, normal cells contain a chemopreventive derivative that facilitates sulfate formation from homocysteine thiolactone. The concentration of this derivative is believed to be diminished during the carcinogenic transformation of normal to malignant cells through the action of carcinogenic chemicals, radiation, microbes or chronic inflammation, as reported by McCully K S in Annals of Clinical and Laboratory Science 2018; 48:386-393. The function of this chemopreventive derivative in normal cells is to prevent accumulation of homocysteine thiolactone by catalyzing its conversion to phosphoadenosine phosphosulfate, sulfate esters of glycosaminoglycans, steroids, and other compounds, and sulfate ions. Decreased concentration of this chemopreventive derivative in malignant cells leads to the characteristic metabolic abnormalities of malignancy, which are attributable to excessive accumulation of homocysteine thiolactone. According to this concept, the increased growth rate, the aggregation of nucleoproteins, the increased expression of developmentally suppressed genes, the degradation of cellular membranes, and the abnormalities of oxidative metabolism, such as aerobic glycolysis, are attributable to increased accumulation of homocysteine thiolactone within malignant cells. Treatment of animals with transplanted malignant neoplasms by homocysteine thiolactone perchlorate causes increased necrosis within malignant neoplasms, presumably by increased accumulation of homocysteine thiolactone within malignant tissues, as taught in U.S. Pat. No. 4,255,443.

The identity of the N-substituted derivative of homocysteine thiolactone that prevents growth of malignant tumors in animals was elucidated by organic synthesis of anti-neoplastic compounds containing homocysteine thiolactone. Arachidonoyl homocysteine thiolactone amide and pyridoxal homocysteine thiolactone enamine decrease the growth of transplanted murine mammary adenocarcinoma, as reported by McCully K S et al in Chemotherapy 1977; 23:44-49. As taught in U.S. Pat. No. 4,383,994, N-maleyl homocysteine thiolactone amide, N-maleamide homocysteine thiolactone amide, and rhodium trichloride oxalyl homocysteine thiolactone amide suppress the growth of transplanted neoplasms in animals. Encapsulation of N-maleamide homocysteine thiolactone amide within liposomes greatly enhances its anti-neoplastic activity, as reported by McCully K S et al in Proceedings of the Society for Experimental Biology and Medicine 1985; 180:57-61. Structural analysis of these biologically active derivatives of homocysteine thiolactone shows that the chemopreventive derivative of homocysteine thiolactone in normal cells is (1) active in a lipid-soluble form, (2) contains a conjugated double bond system with a carbonyl group adjacent to the nitrogen atom of homocysteine thiolactone, and (3) forms a complex with a transition metal atom that enhances anti-neoplastic activity.

U.S. Pat. Nos. 4,618,685 and 4,925,931 teach that the reaction of homocysteine thiolactone with retinoic acid forms N-homocysteine thiolactonyl retinamide (TR), known as thioretinamide, and thioretinamide reacts with cobalamin to form N-homocysteine thiolactonyl retinamido cobalamin (TR₂Co), known as thioretinaco. Both thioretinamide and thioretinaco have anti-carcinogenic and anti-neoplastic activities, as reported by McCully K S et al in Carcinogenesis 1987; 8:1559-1562 and in Proceeding of the Society for Experimental Biology and Medicine 1989; 191:346-351. The method of synthesis of thioretinamide was significantly improved by use of N-ethyl-N′-(3-dimethyl-aminopropyl) carbodiimide in the reaction mixture, as taught in U.S. Pat. Nos. 6,054,595 and 6,287,818. This method replaces the conjugation agent, dicyclohexylcarbodiimide in the reaction mixture of the original method and produces pure thioretinamide in 72% of theoretical yield. This pure thioretinamide and its complex with cobalamin, thioretinaco, have anti-atherogenic activity in rats treated with parenteral homocysteine thiolactone, as reported by Kazimir M et al in Research Communications in Molecular Pathology and Pharmacology 2002; 5,6:179-198.

As taught in U.S. Pat. No. 5,565,558 the anti-carcinogenic, anti-neoplastic, anti-viral, and anti-aging activities of thioretinaco ozonide are enhanced by use of membranergic compositions, specifically the polypeptide cytokines, alpha-interferon, beta-interferon, and gamma-interferon. As taught in U.S. Pat. No. 6,696,082 a therapeutically active composition of thioretinaco ozonide for providing anti-carcinogenic, anti-neoplastic, anti-viral, anti-atherogenic, and anti-aging benefits is formed by thioretinaco ozonide, complexed with adenosine triphosphate and oxygen within an ozone-resistant liposomal carrier.

Studies of homocysteine thiolactone metabolism in the liver of scorbutic guinea pigs that are deprived of dietary ascorbate disclosed a failure of oxidation of homocysteine thiolactone to the disulfide, homocystine, and inorganic sulfate, as well as a pathway for synthesis of phosphoadenosine phosphosulfate from the sulfur atom of homocystein thiolactone, as reported by McCully K S in Nature 1971; 231:391-392. Homocysteic acid, the oxidized sulfonic acid derivative of homocysteine, promotes growth in normal animals and promotes growth and release of insulin-like growth factor, IGF-1, in hypophysectomized animals that are treated with thyroxine, as reported by Clopath P et al in Science 1976; 192:372-374. Young animals and hypophysectomized animals convert more homocysteine thiolactone to homocysteic acid and other oxidized homocysteine derivatives than older or normal animals, as reported by McCully K S in Annals of Clinical and Laboratory Science 1975; 5:147-152. Cultured cells that are deficient in cystathionine synthase and unable to convert homocysteine to cystathionine are able to oxidize the sulfur atom of homocysteine thiolactone to sulfate, demonstrating a pathway for sulfate synthesis that is independent of conversion of homocysteine to cystathionine, cysteine and sulfate, as reported by McCully K S in American Journal of Pathology 1972; 66:83-95. The pathway for synthesis of sulfate from homocysteine thiolactone involves synthesis of thioretinamide from homocysteine thiolactone and retinoic acid and subsequent oxidation of thioretinamide to sulfite, alpha-keto-butyrate and retinoic acid by superoxide, as described by McCully K S in Annals of Clinical and Laboratory Science 1994; 24:27-59.

Nutritional studies have demonstrated that the hyperhomocysteinemia of protein energy malnutrition is associated with reduction in levels of plasma transthyretin, the plasma protein that transports retinol binding protein and thyroxine, as reported by Ingenbleek Y et al in Nutrition 2002; 18:40-46. The metabolic disorder caused by protein energy malnutrition involves decreased synthesis and activity of cystathionine synthase, leading to hyperhomocysteinemia and decreased synthesis of cystathionine and cysteine, as reported by Ingenbleek Y et al in Nutrition 2012; 28:148-153. Transthyretin contains abundant tryptophan, and the plasma level of transthyretin declines in protein energy malnutrition because of dietary deficiency of tryptophan and other essential amino acids, leading to decreased endogenous synthesis of transthyretin. The heme oxygenase function of cystathionine synthase catalyzes the generation of superoxide radical from dioxygen, as reported by Carballal S et al in Biochemistry 2008; 47:3194-3201. Retinoic acid enhances the stimulation by thyroid hormone of heme oxygenase activity in the liver of thyroidectomized rats, as reported by Smith J J et al in Biochimica et Biophysica Acta 1991; 1075:119-122, demonstrating interaction between retinoic acid and the heme group of heme oxygenase. N-(4-hydroxyphenyl)-retinamide, known as fenretinide, induces apoptosis in retinal cells through reactive oxygen species generation and through increased expression of heme oxygenase, as reported by Samuel W et al in Journal of Cellular Physiology 2006; 209:854-865. Investigation of fenretinide demonstrates anti-neoplastic potential, because of its ability to induce apoptosis in malignant cells, as discussed by Hail N Jr et al in Apoptoris 2006; 11:1677-1694, and to increase insulin sensitivity in subjects at risk for breast cancer, as discussed by Johannsson et al in Cancer Research 2008; 68:9512-9518.

Taken together, these observations indicate that retinol is delivered to cells by the retinol binding protein component of transthyretin, and the heme oxygenase function of cystathionine synthase is responsible for the oxidation of retinol and the simultaneous reaction of retinoic acid with homocysteine thiolactone to produce thioretinamide. This process is catalyzed by binding of dehydroascorbate to the heme group of cystathionine synthase and production of superoxide radical from dioxygen. In catalyzing this reaction of retinol with superoxide radical, dehydroascorbate is simultaneously reduced to semidehydroascorbate, and thioretinamide is formed by the reaction of homocysteine thiolactone with enzyme-bound retinoic acid. These reactions are illustrated as follows:

retinol+superoxide radical+dehydroascorbate→retinoic acid+semidehydroascorbate+water Reaction 1:

retinoic acid+homocysteine thiolactone→thioretinamide+water Reaction 2:

Thioretinamide is subsequently further metabolized to alph-keto-butyrate, hydrogen sulfide, and retinoic acid, and this pathway is facilitated by oxidation of hydrogen sulfide to sulfite and sulfate by superoxide.

This formulation of the synthesis of thioretinamide from retinol and homocysteine thiolactone by the heme oxygenase function of cystathionine synthase explains the failure of sulfate synthesis from homocysteine thiolactone in experimental scurvy and the function of dehydroascorbate in sulfate synthesis, as reported by McCully K S in Nature 1971; 231:391-392. Thioretinamide is a precursor of thioretinaco by reaction with cobalamin, as taught in U.S. Pat. No. 4,925,931 and reported by McCully K S et al in Proceedings of the Society for Experimental Biology and Medicine 1989; 191:346-351. Thioretinaco ozonide catalyzes the process of oxygen utilization in oxidative phosphorylation, as reported by McCully K S in Annals of Clinical and Laboratory Science 1994; 24:27-59. The synthesis of thioretinaco from thioretinamide is facilitated by thyroxine that is transported by plasma transthyretin, explaining how oxidative metabolism is stimulated by thyroxine. Only higher eukaryotes contain cystathionine synthase with a heme functional group, and the cystathionine synthase of prokaryotes, such as yeast and flagellates, contains no heme functional group, as discussed by Miles E W et al in Journal of Biological Chemistry 2004; 279:29871-29874. Since embryonic and malignant cells are deficient in the activity of cystathionine synthase, as reported by Kim J et al in Oncology Reports 2009; 21:1449-1454, this formulation explains why malignant cells are deficient in oxidation of homocysteine thiolactone to sulfate by the heme oxygenase function of cystathionine synthase.

Human glioma cells transfected with lentiviral vectors encoding shRNA targeting cystathionine synthase were injected into immune-deficient mice, causing a decreased latency period of xenograft growth after subcutaneous and intracerebral injection, as reported by Takano N et al in Molecular Cancer Research, 2014; 12:1398-1406. In addition, knockdown clones of cystathionine synthase-deficient glioma cells display increased anchorage-independent cell growth and higher levels of hypoxia-inducible factor 2a, suggesting that glioma tumor formation is promoted by cellular deficiency of cystathionine synthase. In a study of human gastric cancer and colorectal cancer cells, down-regulation of the mRNA for cystathionine synthase (CBS) is caused by CpG methylation of the CBS gene suggesting that this gene functions as a tumor suppressor gene, as reported by Zhao H et al in Public Library of Science One 2012; 7:e49683. Hypermethylation of DNA is demonstrated in cell culture models of mitochondrial DNA depletion, which is dependent upon NADH oxidation, increased adenosyl methionine, and changes in polyamine synthesis, associated with mitochondrial dysfunction, as reported by Lozoya O A et al in Public Library of Science Biology 2018; 16:e2005707. Oxidized NAD⁺ is a component of the active site of oxidative phosphorylation, thioretinaco ozonide oxygen nicotinamide adenine dinucleotide phosphate (TR₂CoO₃O₂NAD⁺H₂PO₄⁻), as proposed by McCully K S in Annals of Clinical and Laboratory Science 2015; 45:222-225. Thioretinamide and thioretinaco biosynthesis are catalyzed by cystathionine synthase, as reported by McCully K S in Annals of Clinical and Laboratory Science 2011; 41:300-313.

Retinoids and retinoic acid regulate physiological processes by binding to retinoic acid receptors (RAR) and retinoic×receptors (RXR), which constitute a class of DNA-binding transcription regulators, as reviewed by Das B C et al in Bioorganic and Medicinal Chemistry 2014; 22:673-683. Thioretinaco contains two molecules of thioretinamide, which consists of the amide formed from retinoic acid and homocysteine thiolactone, as reviewed by McCully K S in Annals of Clinical and Laboratory Science 1994; 24:27-59. Thus thioretinaco binds to the RXR receptor and participates in formation of the RAR/RXR receptor that regulates the tumor suppressor gene CBS which codes for cystathionine synthase biosynthesis, causing enhanced activity of cystathionine synthase in normal cellular function. Carcinogenesis by chemical carcinogens or by oncogenic viruses and microbes causes loss of the active site of oxidative phosphorylation and mitochondrial dysfunction by decomposition of the thioretinaco disulfonium complex with ozone, oxygen and ATP, as reported by McCully K S in Annals of Clinical and Laboratory Science 2018; 48:386-393. Consequently, loss of cystathionine synthase activity in malignant cells results in decreased activation of the tumor suppressor CBS gene by the RAR/RXR complex formed from thioretinaco ozonide. Hence, repletion of the active site of oxidative phosphorylation by activation of the tumor suppressor CBS gene by the RAR/RXR complex formed from thioretinaco ozonide has the potential for increasing biosynthesis by cystathionine synthase of thioretinaco, which is depleted by opening of the mitochondrial permeability transition pore in aging, cellular senescence and mitochondrial dysfunction, as reviewed by McCully K S in Annals of Clinical and Laboratory Science 2018; 48:In Press. A nutrigenetic study of hyperhomocysteinemia by quantitative proteomics identified the retinoid×receptor signaling pathway as the most prominent network associated with the heterozygous CBS^{+/-} genotype, as reported by DiBello P M et al in Molecular and Cellular Proteomics 2010; 9:471-485.

Experimental evidence for the efficacy of thioretinaco ozonide in suppressing tumor growth is provided by in vivo and in vitro treatment of human pancreatic cancer cells growing in athymic mice or in cell culture. Direct injection of thioretinaco into subcutaneous tumors causes a 50% reduction of tumor weight after 6 weeks of growth in athymic mice, compared with propylene glycol vehicle controls, but no growth inhibition is observed from thioretinamide, adenosyl cobalamin, homocysteine thiolactonyl cobalamin, or homocysteine thiolactone perchlorate, as reported by McCully K S et al in Research Communications in Chemical Pathology and Pharmacology 1989; 66:117-122. Experiments with cultured human pancreatic carcinoma cells revealed inhibition of cellular growth by added thioretinaco or thioretinamide in the culture medium after 4 days, compared with ethanol vehicle controls, as reported by McCully K S et al in Research Communications in Chemical Pathology and Pharmacology 1992; 77:125-128. Presumably, thioretinaco inhibits growth of malignant cells by up-regulating endogenous biosynthesis of thioretinaco ozonide by the action of RAR/RXR complexes of thioretinaco on activation of the CBS tumor suppressor gene for biosynthesis of cystathionine synthase. Only the highest concentrations of thioretinaco decrease growth of cultured normal fibroblasts, consistent with higher concentrations of intracellular thioretinaco within normal cells, compared with malignant cells, the growth of which is inhibited by low concentrations of thioretinaco in the culture medium.

The use of stem cell products in regenerative medicine is the subject of clinical investigation of a wide spectrum of conditions, for example macular degeneration, type 1 diabetes mellitus, heart failure, Parkinson's disease, stroke, amyotrophic lateral sclerosis, glioblastoma, and spinal cord injury, as reviewed by Trounson A et al in Cell Stem Cell 2015; 17:11-22. Mesenchymal stem cells from bone marrow or adipose tissue are the most frequently studied cell type, but other cell types, including embryonic stem cells, induced pluripotent stem cells, embryonic neural stem cells, endothelial progenitor cells, placental mesenchymal stem cells, and limbal stem cells are also the subject of clinical trials. There have been few reports of safety issues from stem cell therapy, and the effect on numerous tumor biomarker levels of multiple intravenous administrations of cultured human autologous adipose-derived mesenchymal stem cells reveal no significant changes in tumor biomarkers, irrespective of gender and age, as reported by Ra J C et al in Journal of Clinical Case Reports 2017; 7:10001040. Adipose-derived mesenchymal stem cells are effective in delivering a gene for cytosine deaminase to colonic tumor cells in culture or in athymic mice, and a migratory effect toward some tumor types is demonstrated, as reported by Kucerova L et al in Cancer Research 2007; 67:6304-6313. Human embryonic stem cells have not frequently been utilized in stem cell therapy because of ethical and legal considerations.

Because of the ethical and legal issues surrounding clinical use of human embryonic stem cells, adult stem cells derived from the mesenchymal component of bone marrow, have been characterized extensively in the prior art. Because of the paucity of mesenchymal stem cells in bone marrow, difficulty of isolation, and declining number of bone marrow cells with aging, human adipose tissue has been identified as a satisfactory alternative source of human adult mesenchymal stem cells for use in clinical therapy, as reviewed by Zuk P A et al in Molecular Biology of the Cell 2002; 13:4279-4295. Mesenchymal stem cells isolated from human lipoaspirates of adipose tissue differentiate in vitro toward osteogenic, adipogenic, myogenic, neurogenic, and chondrogenic lineages when treated with established lineage-specific factors, as demonstrated by the CD marker profile, and by lineage-specific enzymatic and signaling markers. These results identify human adult adipose tissue as an important source of pluripotent mesenchymal stem cells with multi-germline potential.

A comparative analysis of human adult mesenchymal stem cells isolated from bone marrow, umbilical cord blood, or adipose tissue studied the morphology, success of isolation, colony frequency, expansion potential, and immune phenotype of these three types of stem cells, as reported by Kern S et al in Stem Cells 2006; 24:1294-1301. While no differences in morphology or immune phenotype were identified, success rate of isolation was 100% for bone marrow and adipose sources, but only 63% for umbilical cord blood stem cells. Umbilical cord blood stem cells have the highest proliferation capacity, but no adipogenic differentiation capacity, in contrast to bone marrow and adipose mesenchymal stem cells which have multi-lineage differentiation capacity. Both umbilical cord blood and adipose tissue are preferable alternatives to bone marrow in isolating mesenchymal stem cells, as adipose tissue contains the highest frequency and cord blood is expandable to higher numbers of mesenchymal stem cells. Umbilical cord blood stem cells have the highest rate of senescence, and bone marrow stem cells also have a high senescence rate in early passages, whereas adipose stem cells have the lowest rate of senescence.

The effect of donor age on the function of induced pluripotent stem cells was reported by Strassler et al in Frontiers in Cardiovascular Medicine 2018; 5:4. Induction of pluripotent stem cells from cultured fibroblasts is accomplished by exposure to the signaling factors, Sox-2, Oct-3/4, Klf-4 and c-Myc, using retroviral vectors and more recently adenovirus, Sendai virus, and recombinant proteins. Because of telomerase activation, changes in methylation of DNA and mitochondrial morphology, reduced markers of senescence like p21 in induced pluripotent stem cells, propagation is enhanced indefinitely by cellular re-programming. Thus markers of cellular aging are reverted in the process of induced pluripotent stem cell reprogramming, and both differentiation potential and early senescence are unrelated to donor age. However, the potential tumorigenicity of these cells and the impact of epigenetic pattern retention are presently unknown.

Stem cells have been utilized for regenerative therapy for cardiac diseases, including heart failure, myocardial infarction, and cardiomyopathy, as reviewed by Ptaszek L M et al in Lancet 2012; 379:933-942. Therapy with bone marrow stem cells revealed minor improvements in outcome in cardiac diseases that was attributed to paracrine factors rather than engraftment of stem cells with cardiomyocytes. Therapy of cardiac diseases with adipose-derived mesenchymal stem cells is the subject of current clinical trials. Delivery of regenerative therapy by mesenchymal stem cells for cardiac disease is compromised by inefficient cellular retention in animal models.

Bone marrow stem cells function as donors of renal progenitor cells, contributing to the formation of mesangial cells, tubular epithelial cells, and podocytes in acute renal failure in animal models, as reviewed by Yokoo T et al in Organogenesis 2007; 3:34-43. Stem cell therapy of chronic renal failure involves attempts at organogenesis of a neo-kidney from embryonic metanephros in organ cultures of animal models. In a study of fetal retinal implants in patients with macular degeneration or retinitis pigmentosa, phase 2 clinical trials documented improvements in vision, corroborating animal models of retinal degeneration, as reviewed by Daftarian N et al in Journal of Ophthalmic and Vision Research 2010; 5:250-264. Induced pluripotent stem cells (iPS) reprogrammed from somatic cells have the capacity to differentiate into keratinocytes, providing a novel approach to stem cell therapy of cutaneous diseases such as epidermolysis bullosa, as reviewed by Uitto J in Journal of Investigative Dermatology 2011; 131:812-414.

In experiments with human glioma intracranial xenografts in immune-deficient mice, human mesenchymal cells (hMSC) isolated from bone marrow of normal human volunteers were labelled with fluorescent markers and injected directly into ipsilateral or contralateral carotid artery, as reported by Nakamizo A et al in Cancer Research 2005; 65:3307-3318. The labelled hMSC were demonstrated exclusively within brain tumors, regardless of whether the cells were injected into ipsilateral or contralateral carotid arteries. In contrast to hMSC, injection of labelled fibroblasts or labelled human glioma cells resulted in widespread distribution of delivered cells without tumor specificity. Injection of hMSC into the cerebral hemisphere opposite from the hemisphere containing an established human glioma xenograft, demonstrates the ability of hMSC to migrate into the glioma xenograft in vivo, regardless of the site of injection. Engineered hMSC containing interferon-β are demonstrated to increase the lifespan of animals containing human glioma xenografts, suggesting that the tropism of hMSC for human gliomas offers a potential function of hMSC containing a therapeutic agent.

Human tumor necrosis factor-related apoptosis-inducing ligand (TRAIL) induces apoptosis in a variety of human tumors but spares normal tissues, and human adipose-derived mesenchymal stem cells (AD-MSC) containing TRAIL are demonstrated to inhibit growth of a variety of cultured human tumor cell lines, as reported by Grisendi G et al in Cancer Research 2010; 70:3718-3729. Subcutaneous or intravenous injection of AD-MSC containing TRAIL into immune-deficient mice causes localization within xenografts of human tumors, mediating apoptosis of tumors without toxicity to normal tissues. The results are interpreted to support the use of adipose-derived mesenchymal progenitors as cellular carriers containing a therapeutic agent.

In a study of human meduloblastoma, atypical teratoid/rhabdoid tumor, and glioblastoma, tumor spheres and cultured tumor cells were isolated, and human adipose derived stem cells (AD-MSC) are demonstrated to migrate within tumor spheres and cultured tumor cells using an in vitro migration assay and in vivo bioluminescence imaging analysis, as reported by Choi S A et al in Public Library of Science One 2015; 10:e0129292. In this study mRNAs for cytokine/chemokine receptors are inhibited using siRNAs in the different tumor types, demonstrating the migratory ability of AD-MSC for tumor spheres and implying the potential use of human adipose derived stem cells as carriers for gene therapy or other therapeutic agents.

Hyperhomocysteinemia is an independent risk factor for development of atherosclerosis, and loss of thioretinaco ozonide from mitochondria is implicated in the origin of hyperhomocysteinemia and mortality, as reviewed by McCully K S in Annals of Clinical and Laboratory Science 1993; 23:477-493, 2009; 39:219-232 and in Comprehensive Physiology 2016; 6:471-505. Thioretinaco prevents experimental atherogenesis produced in rats by administration of homocysteine thiolactone, as reported by Kazimir M et al in Research Communications in Molecular Pathology and Pharmacology 2002; 111:179-198. Exposure of cultured endothelial cells to homocysteine causes accelerated cellular senescence, as assayed by shortening of telomere length and increased activity of acidic β-galactosidase, as reported by Xu D et al in FEBS Letters 2000; 470:20-24. Endothelial progenitor cells are stem cell precursors of mature endothelial cells, and decreased numbers of endothelial progenitor cells are observed in subjects with increased risk of cardiovascular disease, as reported by Vasa M et al in Circulation Research 2001; 89:e1-e7. Exposure of cultured human blood mononuclear cells to homocysteine decreases the number and the adhesive, migratory and vasculogenic activities of endothelial progenitor cells, as reported by Chen Z J et al in Journal of Molecular and Cellular Cardiology 2004; 36:233-239. Exposure of cultured endothelial progenitor cells to homocysteine decreases proliferation and accelerates the onset of cellular senescence, associated with diminished telomerase activity and Akt phosphorylation, as reported by Zhu J H et al in Journal of Molecular and Cellular Cardiology 2006; 40:648-652.

Aging, cellular senescence and mitochondrial dysfunction are attributed to loss of the active site of oxidative phosphorylation, TR₂CoO₃O₂NAD⁺H₂PO₄⁻, from mitochondria by opening of the inner membrane permeability pore, as reviewed by McCully K S in Annals of Clinical and Laboratory Science 2018; 48:In Press. Plasma homocysteine levels are associated with all-cause mortality, as demonstrated by a dose-response meta-analysis of prospective studies, as reported by Fan R et at in Scientific Reports 2017; 7:4769. Increased plasma homocysteine levels have been associated with macular degeneration, cognitive decline, atherosclerosis and dementia as reviewed by McCully K S in Frontiers in Aging Neuroscience 2017; 9:324. As will be described hereinafter in greater detail in the following Summary of The Invention activated stem cells having an active site of oxidative phosphorylation, TR₂CoO₃O₂NAD⁺H₂PO₄⁻, provides a therapeutic carrier for conversion of the malignant cellular phenotype to a benign cellular phenotype, for prevention of hyperhomocysteinemia and mortality, and for producing decreased cellular senescence and extension of lifespan and the treatment of disease. In addition, this approach provides for the reduction of hyperhomocysteinemia, enhanced immunity and restoration of oxidative metabolism, causing reduction of microbial invasion, and leading to reduction of autoimmune diseases, macular degeneration, and cognitive decline in atherosclerosis and dementia.

### SUMMARY OF THE INVENTION

My invention consists of administration of novel modified or activated stem cell compositions containing the active site of oxidative phosphorylation, TR₂CoO₃O₂NAD⁺H₂PO₄⁻, within the modified stem cell such as mesenchymal stem cells to subjects with mitochondrial deficiency of the active site, causing beneficial therapy of atherosclerosis, cancer, dementia, autoimmunity and diseases of aging. Mesenchymal stem cells, which are derived from human adipose tissue, human bone marrow, or induced human pluripotent cells, migrate into malignant tissues and tissues with mitochondrial deficiency of the active site, preferentially supplying an increased concentration of the active site within target tissues. Culture of mesenchymal stem cells in media containing TR₂CoO₃O₂NAD⁺H₂PO₄⁻ increases the concentration of the active site within mitochondria and supplies an increased concentration of the active site to target tissues when administered intravenously or by direct injection. Delivery of increased concentrations of the active site within stem cells causes repletion of the active site, which is depleted from mitochondria during the pathogenesis of atherosclerosis, cancer, dementia, autoimmunity and diseases of aging, providing a beneficial therapeutic transformation of a malignant cellular phenotype to a benign cellular phenotype, prevention of hyperhomocysteinemia and mortality, enhancement of immunity, and a decrease of cellular senescence, thereby increasing lifespan and providing beneficial therapeutic effects.

The preparation of the novel stem cell compositions having an increased site of oxidative phosphorylation is achieved by incubation of stem cells, such as human mesenchymal stem cells, in a culture medium with about 30 mg/dL of thioretinaco (TR₂Co) with about 30 mg/dL of nicotinamide dinucleotide phosphate (NAD⁺H₂PO₄⁻) exposed to an ozone (O₃) atmosphere of about 1-10% by volume at 37 degrees centigrade until a 90% confluency is obtained.

### DETAILED DESCRIPTION OF THE INVENTION INCLUDING BEST MODE

My invention pertains to the use of human mesenchymal stem cells as a carrier for increased utilization of thioretinaco ozonide oxygen nicotinamide adenine dinucleotide, TR₂CoO₃O₂NAD⁺H₂PO₄⁻, the active site of oxidative phosphorylation within mitochondria, in prevention and treatment of cancer, arteriosclerosis, dementia, autoimmune diseases, and other degenerative diseases of aging. These diseases are all characterized by an abnormality of methionine metabolism in which an increased concentration of homocysteine is demonstrated by assaying plasma or other body fluids for homocysteine bound to proteins by disulfide bonds.

The abnormality of methionine metabolism in diseases of aging is caused by loss or depletion of thioretinaco ozonide oxygen nicotinamide adenine dinucleotide from mitochondria of the cells of the body during aging and disease. Inadequate oxidation of the retinol which is transported to cells and tissues of the body by retinol binding protein and transthyretin, leads to decreased superoxide production by the heme oxygenase function of cystathionine synthase and leads to decreased endogenous synthesis of thioretinamide from retinoic acid and homocysteine thiolactone. The resulting decrease in concentration of cellular thioretinamide leads to decreased production of thioretinaco and thioretinaco ozonide from thioretinamide, cobalamin and ozone. As a result, oxidative phosphorylation is inhibited because of cellular deficiency of thioretinaco ozonide, leading to accumulation of toxic free radical compounds and producing oxidative stress. In addition, the decreased biosynthesis of thioretinaco ozonide leads to increased production of homocysteine thiolactone from methionine and increased homocysteinylation of the free amino groups of proteins, deoxyribonucleic acids, ribonucleic acids, glycosaminoglycans and other macromolecules containing free amino groups by excess homocysteine thiolactone, impairing cellular function and causing accelerated aging of cells and tissues, contributing to the pathogenesis of degenerative diseases of aging.

My invention overcomes the problems with metabolic regulation of oxidative stress in human disease of the prior art by utilization of modified or activated stem cells and in the preferred embodiment human mesenchymal stem cells as a carrier having an active site of oxidative phosphorylation to enhance the endogenous biosynthesis of thioretinamide and thioretinaco by cystathionine synthase within cells and tissues, thereby stimulating cellular oxidative metabolism and reducing the endogenous accumulation of reactive oxygen species and reducing the degradation of cellular and tissue constituents by free radical substances in chronic degenerative diseases. Enhanced biosynthesis of cystathionine synthase results from increased messenger RNA caused by activation of the tumor suppressor gene, CBS by the RAR/RXR complex formed from thioretinaco ozonide. As used herein the terms modified stem cell(s) or activated stem cell(s) refers to a stem cell which has been changed to have an increased active oxidative phosphorylation site having a concentration of TR₂CoO₃O₂NAD⁺H₂PO₄⁻ of approximately 10-300 μg/g.

My invention overcomes the problems with metabolic regulation of oxidative stress in human disease of the prior art by a novel method of increasing the intracellular concentration of the active site of oxidative phosphorylation, TR₂CoO₃O₂NAD⁺H₂PO₄⁻, by delivering this active site complex within mesenchymal stem cells to target cells and tissues by intravenous or direct injection, thereby stimulating cellular oxidative metabolism and reducing the endogenous accumulation of reactive oxygen species and reducing the degradation of cellular and tissue constituents by free radical substances in chronic degenerative diseases. In addition, my invention diminishes the overproduction of homocysteine thiolactone by increasing its conversion to cysteine, metabolites of cysteine, and sulfate, preventing the deleterious homocysteinylation of macromolecules that is characteristic of degenerative diseases and aging. Thus my invention decreases the concentration of homocysteinylated macromolecules, such as homocysteinylated deoxyribonucleic acid and ribonucleic acid, homocysteinylated enzymes and other proteins, and homocysteinylated glycosaminoglycans by increasing conversion of homocysteine thiolactone to sulfate thereby preventing homocysteinylation of macromolecules containing free amino groups, including proteins, nucleic acids, and glycosaminoglycans. In addition, an increased concentration of the active site of oxidative phosphorylation increases endogenous biosynthesis of cystathionine synthase, which is necessary for cellular biosynthesis of thioretinaco ozonide by formation of the RXR/RAR complexes that activate the tumor suppressor gene, CBS and increase the messenger ribonucleic acid (mRNA) for cystathionine synthase biosynthesis.

The source of stem cells to function as a carrier for increased utilization of the active site of oxidative phosphorylation, TR₂CoO₃O₂NAD⁺H₂PO₄⁻, depends upon the intended availability of sufficient numbers of stem cells and the clinical purpose of therapy. The possible sources of stem cells for utilization in my invention includes human mesenchymal stem cells from adipose tissue or from bone marrow, umbilical cord blood, induced pluripotent stem cells, human embryonic stem cells, endothelial progenitor cells, embryonic neural stem cells, placental mesenchymal stem cells, and limbal stem cells. Thus mesenchymal stem cells isolated from adipose tissue have the advantage of convenient availability, adequate numbers of cells, multi-lineage differentiation capacity for use in a variety of clinical applications, and decreased senescence in early passages. Mesenchymal stem cells isolated from bone marrow have the advantage of hematopoietic lineage, but their aspiration from bone marrow is limited in quantity, reduced yield in aged subjects, and like hematopoietic stem cells include admixture with peripheral blood. Human embryonic stem cells have potential for a wide variety of clinical applications, but their use is curtailed by ethical and legal considerations. Other sources of stem cells for specialized applications in clinical treatment of scarring lesions, limbal lesions, neurological conditions, spinal cord injury, depend upon availability and efficacy of multi-lineal differentiation capacity of stem cell types.

My invention enhances innate immunity by providing increased intracellular thioretinaco, which has the capacity to bind ozone to form thioretinaco ozonide, thereby preventing damage to regenerative normal cells which utilize thioretinaco ozonide for oxidative metabolism. My invention is also useful in promoting presentation of antigens of malignant cells to dendritic cells for the activation of natural killer cells, which cause apoptosis of malignant cells by production of ozone and other oxygen radicals, activated by singlet oxygen and antibodies that are useful in the immunotherapy of cancer.

The human chronic degenerative diseases that are benefited by my invention include arteriosclerosis, stroke, acute coronary syndrome, peripheral ischemic gangrene, proliferation of malignant cells in leukemia, lymphoma, sarcoma, carcinoma and melanoma, osteoporosis and fracture, dementia and other neurodegenerative diseases, autoimmune diseases such as lupus etythematosus, ulcerative colitis, thyroiditis, rheumatoid arthritis and pernicious anemia, venous thrombosis and pulmonary embolism, retinal vein thrombosis, retinal artery thrombosis, hypothyroidism, accelerated aging organ transplantation with therapeutic immune suppression, protein energy malnutrition, familial or spontaneous amyloidosis, dietary deficiencies of folate, pyridoxal and cobalamin, complications of pregnancy such as pre-eclampsia and placenta previa, and congenital birth defects, including neural tube defects, cleft palate, and congenital heart disease. My invention ameliorates the course of these human diseases by preventing accumulation of homocysteine within affected cells, preventing oxidative stress from free radical accumulation within cells and tissues, preventing accumulation of homocysteinylated macromolecules with impaired function, increasing endogenous production of hydrogen sulfide from homocysteine by the action of cystathionine synthase and cystathionase, thereby increasing apopotosis of malignant cells and decreasing apoptosis of normal cells in diseases of aging.

Preparation of human mesenchymal stem cells containing an increased concentration of the active site of oxidative phosphorylation, TR₂CoO₃O₂NAD⁺H₂PO₄⁻, is accomplished by incubation of stem cells in cell culture medium containing thioretinaco (TR₂Co), 30 mg/dL, and nicotinamide dinucleotide phosphate (NAD⁺H₂PO₄⁻), 30 mg/dL, and exposed to an atmosphere containing ozone (O), 1-10% by volume. Human mesenchymal stem cells are isolated by liposuction or by bone marrow aspiration, followed by collagenase digestion, filtration of cellular debris, centrifugation and adherence of the cellular pellet to the surface of the cell culture dish. Adherent mesenchymal stem cells are maintained in cell culture medium for 4-5 days at about 37 degrees centigrade at atmospheric pressure until 90% confluency is achieved, subsequently passaged by trypsinization four additional times, and washed in buffered saline for use. Aliquots of the mesenchymal stem cells are checked for cell viability, and absence of fungal, bacterial, endotoxin and mycoplasma contamination. Multiple doses of 10⁸ cells are delivered in 100 ml of sterile saline intravenously to achieve a total dose of 2.5×10¹⁰ cells over a period of two years. The final concentration of TR₂CoO₃O₂NAD⁺H₂PO₄⁻ within the activated stem cells is approximately 10-300 μg/g.

On the other hand the quantity of total cobalamin coenzymes within human adult and fetal brain tissue is reported by Zhang Y et al in Public Library of Science One 2016; 1:e0145797. Total cobalamin in young (0-20 yr) adult brain tissue is 8 pmol/mg protein, corresponding to 0.15 μg/g, total cobalamin in older adult (61-80 yr) brain tissue is 0.05 μg/g, and total cobalamin in fetal brain tissue is 0.30 μg/g, assuming protein of 1.5 mg/100 mg of brain tissue. The concentration of TR₂CoO₃O₂NAD⁺H₂PO₄⁻ in unmodified or prior art stem cells is estimated to be approximately 10% of total cobalamin, since the highest concentration of cobalamin coenzyme consists of methyl cobalamin. Thus TR₂CoO₃O₂NAD⁺H₂PO₄⁻ concentrations correspond to approximately 0.015 μg/g of young adult brain tissue, 0.005 μg/g of older adult brain tissue, and 0.03 μg/g of fetal brain tissue. The culture medium to which novel activated or modified mesenchymal stem cells are exposed contains 30 mg/dL of TR₂Co and 30 mg/dL of NAD⁺H₂PO₄⁻, corresponding to 300 μg/g, a concentration which is 10⁴ fold higher than the fetal brain tissue concentration of TR₂CoO₃O₂NAD⁺H₂PO₄⁻. The achieved concentration of TR₂CoO₃O₂NAD⁺H₂PO₄⁻ within cultured and activated or modified mesenchymal stem cells is 10⁴ fold greater than present within isolated mesenchymal stem cells before incubation.

TABLE 1
Plasma homocysteine levels and risk of diseases of aging

| | | | Plasma homocysteine |
|---|---|---|---|
| Disease risk | Gender | Age | (μmol/L) |
| Low | Male | 20-40 | 4-8 |
| Low | Female | 20-50 | 4-8 |
| Mild | Male | 40-60 | 8-12 |
| Mild | Female | 50-60 | 8-12 |
| Moderate | Male | 50-70 | 10-14 |
| Moderate | Female | 60-70 | 10-14 |
| High | Male | 60-80 | 12-20 |
| High | Female | 70-80 | 12-20 |
| Very high | Male | 60-90 | 16-30 |
| Very high | Female | 70-90 | 16-30 |

As demonstrated in Table 1, the risk of degenerative diseases of aging, including vascular disease, neoplastic disease, autoimmune diseases, osteoporosis and fracture, dementia and other neurodegenerative diseases, thrombotic diseases and renal failure, increases with increasing plasma homocysteine levels. Risk increases at an earlier age for males, compared with females. After menopause, however, risk increases in females to attain a similar disease risk, compared with males of the same age. In Example 1, a 75 year old man with stage IV prostate cancer had a plasma homocysteine level of 14.0 μmol/L, corresponding to moderate to high disease risk. After therapy with intravenous activated mesenchymal stem cells containing TR₂CoO₃O₂NAD⁺H₂PO₄⁻ for two years, the homocysteine level was 9.5 μmol/L. In Example 2, a 75 year old woman with macular degeneration and cognitive impairment had a plasma homocysteine level of 15.4 μmol/L, corresponding to high disease risk. After therapy with intravenous activated mesenchymal stem cells containing TR₂CoO₃O₂NAD⁺H₂PO₄⁻ for two years, the homocysteine level was 8.5 μmol/L. In Example 3, a 60 year old man with acute coronary syndrome had a plasma homocysteine level of 15.8 μmol/L, corresponding to high disease risk. After therapy with intravenous activated mesenchymal stem cells containing TR₂CoO₃O₂NAD⁺H₂PO₄⁻ for two years, the homocysteine level was 10.5 μmol/L. In Example 4, a 65 year old male with metabolic syndrome and early renal failure had a plasma homocysteine level of 16.5 μmol/L, corresponding to very high disease risk. After therapy with intravenous activated mesenchymal stem cells containing TR₂CoO₃O₂NAD⁺H₂PO₄⁻ for two years, the homocysteine level was 10.5 μmol/L. In Example 5, a 70 year old man with arteriosclerosis and aortic aneurysm had a plasma homocysteine level of 18.5 μmol/L, corresponding to very high disease risk. After therapy with intravenous activated mesenchymal stem cells containing TR₂CoO₃O₂NAD⁺H₂PO₄⁻ for two years, the homocysteine level was 10.2 μmol/L. In Example 6, a 65 year old woman with stroke had a plasma homocysteine level of 18.0 μmol/L, corresponding to very high disease risk. After therapy with intravenous activated mesenchymal stem cells containing TR₂CoO₃O₂NAD⁺H₂PO₄⁻ for two years, the homocysteine level was 10.5 μmol/L. In Example 7, the addition of activated mesenchymal stem cells containing TR₂CoO₃O₂NAD⁺H₂PO₄⁻ to the culture medium of cultured human adenocarcinoma cells reduced cellular viability to 0.3%, compared with control cultures. In Example 8, a 69 year old man with localized moderately differentiated prostatic adenocarcinoma was treated with intravenous activated mesenchymal stem cells containing TR₂CoO₃O₂NAD⁺H₂PO₄⁻ for two years, causing reduction of the blood PSA to a normal level, disappearance of the primary malignancy on repeat biopsy, and no significant change in the plasma homocysteine level. In Example 9, a 57 year old woman with solitary metastatic colorectal adenocarcinoma of the liver was treated with intravenous activated mesenchymal stem cells containing TR₂CoO₃O₂NAD⁺H₂PO₄⁻ for two years, causing disappearance of the metastasis and no significant change in the plasma homocysteine level. In Example 10, a 18 month old boy with high risk metastatic neuroblastoma was treated with intravenous activated mesenchymal stem cells containing TR₂CoO₃O₂NAD⁺H₂PO₄⁻ for two years, causing complete regression of metastases and decrease of plasma homocysteine to a normal level for his age.

The novel therapeutic agents and method for utilization of thioretinaco ozonide in activated mesenchymal stem cells as a carrier embodied in my invention are useful for prevention of the induction of malignant neoplasms and for treatment of primary and metastatic neoplasms in human subjects exposed to carcinogenic chemicals, radiation, or oncogenic microbes, causing regression of malignant cell proliferation. The novel therapeutic agents and method of my invention are also useful in prevention and regression of arteriosclerotic plaques of aorta and peripheral arteries in human subjects exposed to an atherogenic diet and multiple infections of the plaques by pathogenic micro-organisms. The novel therapeutic agents and method of my invention are also useful for prevention and regression of cerebral plaques and tangles within neurons in subjects with dementia that are exposed to multiple infections by pathogenic micro-organisms. The therapeutic agents and method of my invention are also useful in prevention of the replication of pathogenic viruses to prevent or cause regression of the pathogenic effects of these viruses and to prevent post-infection sequelae of these viruses. The therapeutic agents and method of my invention are also useful in preventing the degenerative aging changes of their tissues, decreased oxidative metabolism, and decreased life expectancy associated with aging by prevention of further degenerative changes of tissues associated with aging, by enhancement of oxidative metabolism, and by prolongation of life span. In this respect the therapeutic agents and method of my invention are non-toxic and do not suffer the drawback of many known anti-neoplastic, anti-atherogenic, anti-viral, and anti-aging agents, which have cumulative toxic effects after prolonged administration.

Currently available therapies for acute coronary syndrome are not totally effective in preventing recurrent adverse vascular disease events. Current therapies with anti-platelet agents, beta-blockers, anticoagulants, thromboplastin activators, calcium channel inhibitors, and angiotensin converting enzyme inhibitors are only partially effective in therapy and have attendant side effects. Treatment of the hyperhomocysteinemia associated with arteriosclerosis with pyridoxal, folate, and cobalamin does not prevent recurrence of adverse vascular events in subjects with advanced cardiovascular, cerebrovascular, or peripheral vascular disease. Treatment of subjects with early cognitive decline with pyridoxal, folate and cobalamin prevents shrinkage of cerebral tissue susceptible to dementia. However, therapy to remove amyloid deposits from cerebral plaques and intracellular tangles is ineffective in preventing further cognitive decline and dementia. In contrast, treatment of human subjects with the novel activated stem cell compositions and method of my invention will correct the underlying metabolic abnormality leading to acute coronary syndrome and other forms of vascular disease, including vascular dementia, by restoring depleted concentrations of thioretinamide and thioretinaco ozonide within mitochondrial membranes of vascular cells and neurons, restoring endothelial function, preventing a prothrombotic state, and restoring nitric oxide function.

The hyperhomocysteinemia that is characteristic of acute coronary syndrome, metabolic syndrome, chronic arteriosclerosis, and dementia will be prevented by restoration of mitochondrial thioretinaco ozonide, preventing vascular injury, endothelial dysfunction, progression of arteriosclerotic plaques, progression of cerebral plaques and tangles, and recurrent adverse vascular events, such as coronary thrombosis, myocardial infarction, cerebrovascular thrombosis, cerebral Infarction, and ischemic gangrene of the extremities.

The novel therapeutic agents and method of utilization of thioretinaco ozonide oxygen nicotinamide adenine dinucleotide of my invention are deemed useful in preventing the occurrence of spontaneous human neoplasms, including, but not limited to, cancer of lung, skin, colon, breast, prostate, pancreas, brain, lymph nodes, liver, kidney or other organs that arise because of exposure to carcinogenic chemicals, electromagnetic radiation, radiation from radioactive elements, viruses, micro-organisms, inflammatory cytokines, dietary factors, or genetic factors. My invention is deemed useful for the treatment of human neoplasms, causing regression of or preventing metastasis of malignant neoplasms. This invention is useful in treatment of human atherosclerosis, involving aorta, coronary, renal, peripheral, cerebral or other major arteries, causing regression of and prevention of progression of arteriosclerotic plaques, thereby preventing or ameliorating coronary heart disease, stroke, renovascular disease, and peripheral vascular disease. My invention is also useful in treatment of human pathogenic virus infections, including, but not limited to hepatitis virus, immune-deficiency virus, hemorrhagic fever viruses, encephalitis viruses, influenza virus, rhinoviruses, pox viruses, herpetic viruses, and enteric viruses, by preventing viral replication and spread of the virus infection within the cells of the various tissues of the body. My invention is also useful in treatment of human degenerative diseases associated with aging, including, but not limited to, osteoarthritis, osteoporosis, cataract, macular degeneration, dementia, diabetes mellitus, metabolic syndrome, rheumatoid arthritis, thyroiditis, lupus erythematosus, pernicious anemia, and other autoimmune disorders, causing remission or preventing of progression of these diseases within the tissues of the body. It is expected that my invention will be useful in prolonging human life span by preventing degenerative diseases of aging, including atherosclerosis, cancer, autoimmune diseases, and age-associated loss of function of brain, heart, lungs, liver, kidneys, eyes, ears, and other major organs.

The advantages of my invention, as well as aspects of the preferred embodiments, are illustrated more fully in the following Examples:

### Example 1

A 75 year old man was evaluated for treatment of metastatic prostate cancer. The prostate specific antigen (PSA) of blood was determined at 8.0 ng/mL, and needle biopsy demonstrated moderately differentiated adenocarcinoma, Gleason grade 3+3-6/10. Following radical prostatectomy, the PSA value was 0.1 ng/mL and gradually increased to 8.0 ng/mL over a period of 2 years. The plasma homocysteine was initially 10.8 μmol/L, gradually rising to 14.0 μmol/L over a period of 2 years. Computerized tomography scan demonstrated enlarged retroperitoneal lymph nodes, and biopsy of the prostatic surgical site demonstrated recurrent adenocarcinoma. The bone scan revealed no evidence of metastasis. Following luprolide therapy, the PSA value declined to 0.1 ng/mL over a period of three months. To prevent metastasis and hormone resistance of the adenocarcinoma, luprolide therapy was discontinued, and intravenous activated stem cell therapy containing TR₂CoO₃O₂NAD⁺H₂PO₄⁻ was employed. After two years of therapy, the PSA value was 0.1 ng/mL, the plasma homocysteine was 10.4 μmol/L, and the repeat CT scan and bone scan showed no evidence of lymphadenopathy or bone metastasis. At a subsequent visit 5 years later, the PSA value was 0.1 ng/mL, the plasma homocysteine was 9.8 μmol/L, and no weight loss or pain were reported.

### Example 2

A 75 year old woman was evaluated for treatment of macular degeneration and mild recent memory loss. Three years previously decreased vision was noticed in the left eye, and ophthalmological evaluation revealed early supranuclear cataracts bilaterally with edema of the macular area on the left, associated with drusen and retinal pigment epithelium changes. The plasma homocysteine level was 15.4 μmol/L, and the plasma hs-C-reactive protein (CRP) was 3.2 μmol/mL. The Mini Mental State Examination (MMSE) value was 26.6/30, revealing mild cognitive impairment. The woman never smoked, but there was a family history of macular degeneration. After 3 years, she returned with decreased vision in the right eye, and examination revealed macular edema associated with drusen and retinal pigment epithelium changes. Dental examination revealed caries, plaque, and extensive peri-odontitis. To prevent progression of macular changes and decline in mental function, intravenous activated stem cell therapy containing TR₂CoO₃O₂NAD⁺H₂PO₄⁻ was employed for two years, resulting in plasma homocysteine of 10.7 μmol/L, and the CRP of 0.5 μmol/mL The repeat MMSE was 28.5/30. Visual acuity did not change, and examination revealed decreased macular edema bilaterally. Improved memory was reported by her husband. At a subsequent visit 2 years later, the plasma homocysteine was 8.9 μmol/L, the CRP was less than 0.5 μmol/mL, the MMSE was 28.0/30, visual acuity and macular appearance were unchanged.

### Example 3

A 60 year old man was admitted to hospital with intermittent chest pain and shortness of breath. The man was diaphoretic and restless with acute distress. The troponin was 1.5 ng/mL, the white blood cell count was 15,000/mm³, the plasma homocysteine was 15.8 μmol/L, and the CRP was 7.5 μmol/mL. The electrocardiogram demonstrated ST elevation in the precordial leads. A chest X-ray showed early pulmonary edema and congestion of pulmonary arteries. Dental examination revealed caries, plaque and extensive peri-odontitis. After treatment with pain medication, bed rest, and digoxin, the electrocardiogram reverted to normal, his symptoms improved, and after intravenous activated stem cell therapy containing TR₂CoO₃O₂NAD⁺H₂PO₄⁻ was employed for two years, the plasma homocysteine was 9.8 μmol/L, the CRP was 0.5 μmol/mL, and the troponin was undetectable. There was no recurrence of chest pain, and the electrocardiogram was normal. At a subsequent visit 5 years later the plasma homocysteine was 10.5 μmol/L, the CRP was less than 0.5 μmol/L, the troponin was undetectable, the electrocardiogram was normal, and there was no recurrence of chest pain.

### Example 4

A 65 year old male was evaluated for treatment of obesity, hypertension, and elevated blood glucose. During the previous 5 years, gradual weight gain involved abdominal viscera with a protuberant abdomen, the girth increasing to 44 inches. The blood pressure was 180 systolic and 110 diastolic. The plasma homocysteine was 16.5 μmol/L, the fasting blood glucose was 125 mg/dL, the urinalysis revealed microalbuminuria of 2.5 mg/dL, and the plasma creatinine was 2.0 mg/dL. To prevent progression of the metabolic syndrome and early renal failure, intravenous activated stem cell therapy containing TR₂CoO₃O₂NAD⁺H₂PO₄⁻ was employed for two years, resulting in the plasma homocysteine of 11.0 μmol/L, the fasting blood glucose of 98 mg/dL, the urinalysis revealed no protein, and the plasma creatinine was 1.5 mg/dL. Weight loss of approximately 15 pounds was reported, and the abdominal girth measured 41 inches. The blood pressure was 140 systolic and 85 diastolic. At a subsequent visit 2 years later, additional weight loss of 10 pounds and a girth of 40 inches were reported. The plasma homocysteine was 10.2 μmol/L, blood glucose was 95 mg/dL, the urinalysis revealed no protein, and the plasma creatinine was 1.7 mg/dL. The blood pressure was 140 systolic and 85 diastolic.

### Example 5

A 70 year old man with mild abdominal pain was evaluated for treatment of an abdominal aortic aneurysm that was detected by computerized tomography. On examination, a 1 cm ulcer was found on the right great toe. The patient reported the onset of pain in the lower extremities after walking approximately 50 yards. The plasma homocysteine was 18.5 mol/L, the CRP was 10.7 μmol/mL, and the fasting blood glucose was 98 mg/dL. Dental examination revealed caries, plaque, and extensive peri-odontitis. Surgical treatment consisted of excision of the abdominal aortic aneurysm with grafting of the distal aorta, followed by endarterectomy of the right common femoral artery with grafting. The pathology report confirmed the presence of an arteriosclerotic aortic aneurysm, with laminated mural thrombus, and inflammatory changes of the adventitia. Also, fibro-calcific arteriosclerotic plaques were found in the common femoral artery with severe narrowing of the lumen. After recovery from surgery, the ulceration of the great toe gradually healed spontaneously. To prevent progression of generalized arteriosclerosis, intravenous activated stem cell therapy containing TR₂CoO₃O₂NAD⁺H₂PO₄⁻ was employed for two years, resulting in plasma homocysteine of 10.8 μmol/L, the CRP of 1.2 μmol/mL, and the fasting blood glucose of 95 mg/dL. At a subsequent examination two years later there were no further symptoms of abdominal pain, skin ulcers or intermittent claudication.

### Example 6

A 65 year old woman was evaluated for the sudden onset of right sided weakness, associated with inability to speak and difficulty seeing objects in the right visual field. The plasma homocysteine was 18.0 μmol/L, the CRP was 12.5 μmol/mL, and the fasting blood glucose was 102 mg/dL. Following thrombolytic therapy for stroke, her symptoms gradually improved while convalescing at home. After recovering for 3 months, she tripped on a rug and fell, fracturing the right femoral neck. After surgical fixation of the fracture, she was evaluated for osteoporosis that was demonstrated on the X-rays of her fracture site and spine. To prevent further episodes of cerebrovascular disease and fracture, intravenous activated stem cell therapy containing TR₂CoO₃O₂NAD⁺H₂PO₄⁻ was employed. After two years of therapy, the plasma homocysteine was 12.5 μmol/L, the CRP was 4.5 μmol/mL, and the fasting blood glucose was 86 mg/dL. The hemi-paresis and visual field defects were no longer demonstrated. There were no further episodes of mental changes, visual disturbances, or weakness. The hip prosthesis was satisfactory, permitting fill ambulation. At a subsequent evaluation after two years, the plasma homocysteine was 10.5 μmol/L, the CRP was 0.5 μmol/mL, and the fasting blood glucose was 85 mg/dL. There were no further symptoms of weakness, visual disturbances, or mental changes, and ambulation was satisfactory.

### Example 7

Human adenocarcinoma cells were cultured in RPMI medium with added fetal bovine serum and antibiotics, as reported by McCully K S et al in Research Communications in Chemical Pathology and Pharmacology, 1992; 77: 125-128. Equal numbers (10⁵) of adenocarcinoma cells were trypsinized and passaged on day 0, and the cells were refed with media containing test compounds on days 1 and 3. Cell numbers were determined by trypsinization on day 4. Thioretinaco (TR₂Co) and thioretinamide (TR) were synthesized, as previously described (McCully U.S. Pat. No. 4,618,685; McCully U.S. Pat. No. 4,925,931) and dissolved in absolute ethanol. Propylene glycol (PG) was added to the ethanol solutions of the test compounds, the ethanol was evaporated under reduced pressure at 37° C., and the resulting solutions in propylene glycol were added to the culture media. Human mesenchymal stem cells (hMSC) were incubated with cell culture medium containing TR₂Co, NAD⁺ and H₂PO₄⁻ in an atmosphere containing 5% ozone (O₃), to produce activated stem cells and 10⁵ hMSC were added to the culture medium containing adenocarcinoma cells. Equal numbers (10⁵) of hMSC without exposure to TR₂Co, O₃, NAD⁺ and H₂PO₄⁻ served as controls.

TABLE 2
Effect of thioretinaco (TR₂Co), thioretinamide (TR), and human activated
mesenchymal stem cells (hMSC) containing TR₂CoO₃O₂NAD⁺H₂PO₄⁻
on growth of cultured human adenocarcinoma cells.

| | | cell | |
|---|---|---|---|
| | Concentration | growth | cell growth |
| Addition to media | (mg/dL) | (10⁵) | (% of control) |
| None | — | 7.4 ± 2.7 | 100 |
| TR₂Co | 30 | 2.5 ± 0.82 | 33 |
| hMSC | — | 5.0 ± 1.50 | 67 |
| hMSC + | 10⁵ hMSC | 0.3 ± 0.02 | 0.41 |
| TR₂CoO₃O₂NAD⁺H₂PO₄⁻ | | | |
| in 10⁵ hMSC | | | |
| TR | 30 | 1.4 ± 0.50 | 19 |
| PG | 500 | 9.1 ± 0.80 | 123 |

As demonstrated in Table 2, TR₂Co, TR, and activated mesenchymal stem cells (hMSC) containing TR₂CoO₃O₂NAD⁺H₂PO₄⁻ significantly inhibit the growth of cultured human adenocarcinoma cells.

### Example 8

A 69 year old man was evaluated for prostate cancer because of an elevated PSA value of blood of 8.0 ng/mL, and a needle biopsy of prostate demonstrated moderately differentiated adenocarcinoma, Gleason grade 3+2-5/10. Computerized tomography revealed no evidence of enlargement of retroperitoneal lymph nodes. An active surveillance protocol was elected because of the localized nature of the lesion, as described by Wilt T J et al in New England Journal of Medicine 2012; 367:203-213. The plasma homocysteine value was 10.4 μmol/L. Intravenous activated stem cell therapy containing TR₂CoO₃O₂NAD⁺H₂PO₄⁻ was then employed. After two years of therapy, the PSA value was 1.4 ng/mL, and the homocysteine value was 9.8 μmol/L, and a repeat prostate biopsy was negative for adenocarcinoma.

### Example 9

A 57 year old woman was found to have mild iron deficiency anemia, and colonoscopy revealed a rectosigmoid adenocarcinoma. The resection specimen contained a T3N1M0 adenocarcinoma with serosal invasion and metastasis to one of 12 regional lymph nodes. The plasma homocysteine value was 10.5 μmol/mL. After satisfactory recovery, no further therapy was employed. At a subsequent visit 2 years later, mild jaundice was noted, and a liver biopsy of a solitary mass revealed metastatic adenocarcinoma. Intravenous activated stem cell therapy containing TR₂CoO₃O₂NAD⁺H₂PO₄⁻ was employed. After two years of therapy, there was no recurrence of anemia or jaundice, and an ultrasound examination of liver was normal. The plasma homocysteine value was unchanged at 10.5 mol/mL.

### Example 10

An 18 month old baby boy was evaluated for abdominal swelling associated with urinary excretion of dopamine, homovanillic acid, and vanillylmandelic acid. Biopsy of an adrenal mass revealed highly malignant neuroblastoma, bone marrow biopsy revealed infiltration by neuroblastoma cells and scintigraphy with ¹²³I-MIBG (metaiodobenzylguanidine) revealed skeletal metastases. The plasma homocysteine was 18.5 μmol/L. Because of the poor outcome of therapy of ultra-high risk disease, conventional bone marrow transplantation therapy with isotretinoin, as described by Fish J D et al in Bone Marrow Transplantation 2008; 41:159-165, was declined, and experimental intravenous activated stem cell therapy containing TR₂CoO₃O₂NAD⁺H₂PO₄⁻ was employed. After two years of therapy, there was no recurrence of abdominal swelling, and repeat bone marrow biopsy and ¹²³I-MIBG scintigraphy revealed no evidence of bone marrow involvement or skeletal metastases. The plasma homocysteine was 6.8 μmol/L.

My invention is useful in therapy of human diseases of aging characterized by loss of the active site of oxidative phosphorylation, TR₂CoO₃O₂NAD⁺H₂PO₄⁻, from mitochondria, as reflected in mitochondrial dysfunction and elevation of peripheral blood homocysteine concentrations by utilizing a modified stem cell as a carrier for an active oxidative phosphorylation site. From the foregoing description, one skilled in the art can easily ascertain the essential characteristics of my invention and, without departing from the spirit and scope thereof, can make various changes and modifications to adapt my invention to various usages and conditions.

I claim:

1. A therapeutic agent comprising an activated stem cell with an active oxidative phosphorylation site containing thioretinaco ozonide oxygen nicotinamide adenine dinucleotide phosphate.

2. The therapeutic agent of claim 1 wherein the concentration of thioretinaco ozonide oxygen nicotinamide adenine dinucleotide phosphate is from between 10-300 ug/g.

3. The therapeutic agent of claim 1 wherein the activated stem cell is an activated mesenchymal stem cell.

4. The therapeutic agent of claim 2 wherein the activated stem cell is an activated mesenchymal stem cell.

5. The therapeutic agent of claim 1 wherein the activated stem cell is an activated pluripotent stem cell.

6. The therapeutic agent of claim 1 wherein the activated stem cell is an activated embryonic stem cell.

7. The therapeutic agent of claim 1 wherein the activated stem cell is an activated progenitor stem cell.

8. The therapeutic agent of claim 7 wherein the activated progenitor stem cell is an activated endothelial progenitor stem cell.

9. The therapeutic agent of claim 1 wherein the activated stem cell is an activated neural stem cell.

10. The therapeutic agent of claim 1 wherein the activated stem cell is an activated limbal stem cell.

11. The therapeutic agent of claim 1 wherein the activated stem cell is an activated hematopoetic stem cell.

12. The therapeutic agent of claim 3 wherein the activated mesenchymal stem cell is administered parenterally to transform a malignant cell type to a benign cell type.

13. The therapeutic agent of claim 3 wherein the activated mesenchymal stem cell are human activated mesenchymal stem cells administered to treat neuroblastoma.

14. A method of preparing an activated stem cell composition comprising:
(a) harvesting a plurality of stem cells;
(b) incubating in an incubator the plurality of harvested stem cells in a culture medium with thioretinaco (TR₂Co) with nicotinamide dinucleotide phosphate (NAD⁺H₂PO₄⁻) in an ozone atmosphere; and
(c) incubating with heat until about ninety percent (90%) confluency is obtained.

15. The method of claim 14 wherein the incubator is maintained at about 37 degrees centigrade at atmospheric pressure for about 4 to 5 days.

16. The method of claim 15 wherein the plurality of stem cells are passaged by trypsinization and washed in a buffered saline.

17. The product by the process of claim 14.

18. A composition of matter comprising a stem cell activated with about 10-300 μg/g of TR₂CoO₃O₂NAD⁺H₂PO₄⁻ wherein TR₂Co is thioretinaco, O₃ is ozone and O₂ is oxygen and NAD⁺H₂PO₄⁻ is nicotinamide dinucleotide phosphate.

19. The composition of matter of claim 18 wherein the activated stem cell is a mesenchymal stem cell.

20. The composition of matter of claim 18 wherein the activated stem cell is an induced pluripotent stem cell.
